(19) United States
(12) Patent Application Publication
(10) Pub. No.: US 2013/0030009 A1
(43) Pub. Date: Jan. 31, 2013

(54) METHOD FOR THE TREATMENT AND/OR PREVENTION OF ORAL ALLERGIC SYMPTIONS OF THE LIPS DUE TO ORAL CONTACT WITH A FOOD ALLERGEN

(71) Applicants: Ziv Harish, Tenafly, NJ (US); Isaac Rubinstein, Haworth, NJ (US); Russ Weinzimmer, Milford, NH (US); Ehud Arbit, Englewood, NJ (US)

(72) Inventors: Ziv Harish, Tenafly, NJ (US); Isaac Rubinstein, Haworth, NJ (US); Russ Weinzimmer, Milford, NH (US); Ehud Arbit, Englewood, NJ (US)

(21) Appl. No.: 13/365,040

(22) Filed: Feb. 2, 2012

Related U.S. Application Data
(62) Division of application No. 13/026,972, filed on Feb. 14, 2011.
Application No. 13/026,972 is a continuation-in-part of application No. 12/981,726, filed on Dec. 30, 2010, now abandoned.

(51) Int. Cl. A61K 31/352; A61P 37/08; C07D 407/12; A61K 31/495; A61K 31/47

(52) U.S. Cl. 514/255.04; 514/456; 514/311; 549/402

(57) ABSTRACT

A method of applying a topical preparation of at least one of a mast cell stabilizer, an antihistamine, and a leukotriene inhibitor is disclosed for prevention and/or treatment of oral allergy syndrome of the lips, including lip itchiness and/or swelling. For example, topical application of Cromolyn Sodium to the lips can be used to prevent and/or treat allergic reaction to consumption or other contact with raw fruits and/or raw vegetables. The topical administration can be performed by using applicator devices that apply at least one of a mast cell stabilizer and an antihistamine in the form of a liquid, or a gel, or a butter, or a wax-like solid, or a liposome suspension. Applicator devices can include at least one of: a roller, a brush, a sponge, a swab, a tube, a lipstick. The taste of the at least one of a mast cell stabilizer and an antihistamine can be masked by flavors.

### CROSS-REFERENCE TO RELATED APPLICATIONS

This application is a divisional application of U.S. application Ser. No. 13/026,972, filed Feb. 14, 2011 and entitled “APPLICATION TO THE LIPS OF PREPARATIONS FOR TOPICAL PREVENTION AND/OR TREATMENT OF ALLERGIC SYMPTOMS DUE TO ORAL CONTACT WITH FOODS,” which is hereby incorporated herein by this reference in its entirety; which is a continuation-in-part (CIP) of U.S. application Ser. No. 12/981,726, filed Dec. 30, 2010 and entitled “PREPARATIONS FOR TOPICAL PREVENTION AND/OR TREATMENT OF ORAL ALLERGIC SYMPTOMS DUE TO ORAL CONTACT WITH FRUITS AND/OR VEGETABLES,” which is hereby incorporated herein by this reference in its entirety.

### FIELD OF THE INVENTION

This invention relates to prevention and/or treatment of oral allergy symptoms, and particularly to the application of preparations to the lips for topical prevention and/or topical treatment of oral allergy symptoms.

### BACKGROUND OF THE INVENTION

Allergic conditions result from release of mediators, including histamine, from sensitized mast cells upon exposure to an allergen.

Individuals with pollen allergy often experience oral itchiness, i.e., irritation of various surfaces within the mouth and throat, upon ingestion of raw fruits and/or raw vegetables, often resulting in these individuals deciding to avoid eating these foods. The condition of oral itchiness, or other allergic symptom, such as irritation and/or swelling, is the result of local activation of mast cells within tissues of the mouth upon exposure to raw fruits and vegetables that cross-react with the pollens to which the individuals are allergic. As defined herein, “oral itchiness” shall also refer to any allergic symptom affecting the throat (pharynx) and/or the voice box (larynx), such a itchiness, irritation, and/or swelling thereof.

Currently, no approved medical treatment is available to alleviate this condition. Patients are advised to avoid raw fruits to which they are sensitive, but this deprives the patient of important nutrients that are found in fruits and vegetables. Avoidance also causes uncomfortable social problems. There are anecdotal reports of relief with allergy immunotherapy (allergy shots), and with use of antihistamines.

Mast cell stabilizers, such as Cromolyn Sodium, are commonly used in treatment of multiple allergic conditions, including asthma, rhinoconjunctivitis, eczema, and pruritus.
Cromolyn Sodium is a salt of Cromoglicic acid, and is available in multiple forms: as a nasal spray (Rynacrom(UK), Nasalcrom, Prevalin (Netherlands)) to treat allergic rhinitis; in a nebulizer solution for aerosol administration to treat asthma; and as an inhaler (Intal) for preventive management of asthma. The maker of Intal™, King Pharmaceuticals, has discontinued manufacturing the inhaled form, cromolyn sodium inhalation aerosol, due to issues involving CFC-free propellant. As stocks are depleted, this inhaler preparation will no longer be available to patients.
Cromolyn Sodium is also available as eye drops (Opticrom™ and Optrex Allergy™ (UK), Crolom™) for allergic conjunctivitis; and also in an oral form (Gastrocrom™) to treat mastocytosis, dermatographic urticaria and ulcerative colitis. Another oral product, Intercron™ (sodium cromoglicate in distilled water, from Zambon France), is used for systemic treatment of food allergies. However, the physicochemical properties of cromolyn impede its absorption across epithelial lining such as through the oral mucosa and contribute to its poor absorption from the gastrointestinal tract. No more than 1% of a dose administered orally is absorbed by humans, the remainder being excreted in the feces. Very little absorption of Cromolyn Sodium was seen after administration of 500 mg by mouth to each of 12 volunteers. From 0.28 to 0.50% of the administered dose was recovered in the first 24 hours of urinary excretion in 3 subjects. The mean urinary excretion of an orally administered dose over 24 hours in the remaining 9 subjects was 0.45%.

Each 5 mL ampule of Gastrocrom™ contains 100 mg Cromolyn Sodium, USP, in purified water. Cromolyn Sodium, USP, is a hygroscopic, white powder having little odor. It may leave a slightly bitter aftertaste. Gastrocrom™ Oral Concentrate is clear, colorless, and sterile. It is intended for “oral” use, i.e., to be swallowed and reach superficially situated mast cells along the gastrointestinal tract, or to be absorbed systemically via the gastrointestinal tract, just as pills, capsules, and tablets are to be swallowed and absorbed systemically.

In particular, users of Gastrocrom™ are instructed to: Squeeze liquid contents of an ampule containing a single dose of Gastrocrom™ into a glass of water. Stir the resulting solution. Drink all of the solution immediately.

Also, Cromolyn Sodium is commonly administered via a power-driven nebulizer, under the brand names: Intal™ and Intal Inhaler™. The nebulizer must have a suitable face mask or mouthpiece. Nebulizers make a liquid into an aerosol that one breathes in through one's mouth, or through one's mouth and nose, into the lungs. However, one is also commonly advised: “Do not take this medicine as a solution by mouth”. One is also advised “If you get a bitter or unpleasant taste in your mouth, gargle or rinse your mouth after you use this medicine.” Patients are also advised: “To relieve dry mouth or throat irritation caused by cromolyn inhalation, rinse your mouth with water, chew gum, or suck sugarless hard candy after each treatment.”

Patients are advised to: “Use cromolyn sodium inhalation on a regular basis for best results. It may be several weeks before you see the effects.” Patients are further advised: “It is very important that you use your cromolyn sodium inhaler properly, so that the medicine gets into your lungs. Your doctor may want you to use a spacer with your inhaler.” Cromolyn oral inhalation will no longer be available in the U.S. after Dec. 31, 2010.

Stated disadvantages include: “Cromoglicic acid requires administration four times daily, and does not provide additive benefit in combination with inhaled corticosteroids.”

It is commonly accepted that Cromolyn Sodium is poorly absorbed topically through mucosal barriers, and is consequently not absorbed well into the oral mucosa. Further, it is also commonly believed that the physicochemical properties including the polarity of the Cromolyn Sodium molecule may also interfere with its absorption and local activity.

In the co-pending patent application “PREPARATIONS FOR TOPICAL PREVENTION AND/OR TREATMENT OF ORAL ALLERGIC SYMPTOMS DUE TO ORAL CONTACT WITH FRUITS AND/OR VEGETABLES”, filed Dec. 30, 2010 application Ser. No. 12/981,726, a topical preparation of at least one of a mast cell stabilizer and/or an antihistamine is disclosed for prevention and/or treatment of oral allergy syndrome, or other allergy-related oral inflammatory processes. For example, the topical administration of Cromolyn Sodium prior to ingestion of raw fruits and raw vegetables may be used to prevent oral allergy symptoms. The topical administration can be performed by use of a solution, a powder, an aerosol, a tablet, a chewable tablet, a gel, a melting strip, a mouth rinse, a mouth swab, or a piece of candy, and may be combined with carrier molecules and/or mucoadhesive molecules to enhance efficacy.

Using the teachings of the co-pending patent application entitled “PREPARATIONS FOR TOPICAL PREVENTION AND/OR TREATMENT OF ORAL ALLERGIC SYMPTOMS DUE TO ORAL CONTACT WITH FRUITS AND/OR VEGETABLES”, filed Dec. 30, 2010, application Ser. No. 12/981,726, a number of patents afflicted with oral allergy syndrome were treated with Cromolyn Sodium mouth rinse. These patients experienced much relief of their oral allergy and throat allergy symptoms. However, some individuals still experienced lip itchiness, of both the internal and external lip surfaces. Thus, it seems that sometimes mouth rinsing of Cromolyn Sodium fails to deliver sufficient Cromolyn Sodium to the mast cells of the lip.

### SUMMARY OF THE INVENTION

A general aspect of the invention is a method for the treatment and/or prevention of oral allergic symptoms of the lips due to oral contact with a food allergen. The method includes topically administering to the lips of an effective amount of at least one of: a mast cell stabilizer; an antihistamine; and a leukotriene inhibitor.

In some embodiments, the mast cell stabilizer also has anti-histamine properties.

In some embodiments, to extend contact time of the at least one of the mast cell stabilizer and the antihistamine with the lip, at least one of the mast cell stabilizer and the antihistamine is mixed with at least one of: an oily carrier and mucoadhesive material.

In some embodiments, the mast cell stabilizer is Cromolyn Sodium.

In some embodiments, the mast antihistamine is levocetirizine.

In some embodiments, the leukotriene inhibitor is montelukast.

In some embodiments, the mast cell stabilizer is mixed with a carrier molecule so as to enhance local absorption.

In some embodiments, an effective amount of at least one of a mast cell stabilizer and an antihistamine is applied by at least one of: a lip swab, a lipstick, a lip roller, a lip brush, a sponge, a tube, a jar.

Another general aspect of the invention is the use of a mast cell stabilizer in the manufacture of a medicament for the topical treatment and/or topical prevention of oral allergic symptoms of the lips.

Another general aspect of the invention is the use of an antihistamine in the manufacture of a medicament for the topical treatment and/or topical prevention of oral allergic symptoms of the lips.

Another general aspect of the invention is the use of a leukotriene inhibitor in the manufacture of a medicament for the topical treatment and/or topical prevention of oral allergic symptoms of the lips.

Another general aspect of the invention is a pharmaceutical formulation for use in the prevention and/or treatment of oral allergic symptoms of the lips of a person. The pharmaceutical preparation includes an effective amount of at least one of: a mast cell stabilizer and an antihistamine, the effective amount being effective for treatment and/or prevention of oral allergic symptoms of the lips of a person.

In some embodiments, the mast cell stabilizer is selected from: Cromolyn Sodium, Cromoglicate (cromolyn), Nedocromil, Pemirolast potassium, Nedocromil sodium, Lodoxamide tromethamine, Bepotastine besilate, Epinastein HCl, Azelastine HCl, Olopatadine HCl, Ketotifen fumarate.

In some embodiments, the antihistamine is selected from: first generation antihistamines, second generation antihistamines, third generation antihistamines.

In some embodiments, thje oral allergic symptoms of the lips of the person are due to the consumption of at least one of: a piece of raw fruit, a piece of raw vegetable, a nut, fish, shellfish, dairy, baked goods, eggs, soy, wheat, corn.

In some embodiments, the pharmaceutical formulation further includes a delivery medium including at least one of: a thickening agent, a viscous agent, a wetting agent, an adhesive agent, a flavored solution, a taste-masking compound, a lip-gloss color base.

In some embodiments, the pharmaceutical formulation is delivered to oral mucosa of the lips of the person as one of: a liquid, a liposome suspension, a gel, a butter, a wax-like solid.

Another general aspect of the invention is an applicator device for treatment of oral allergy symptoms of the lips. The applicator device includes: a solution of at least one of: a mast cell stabilizer, a leukotriene inhibitor, and an antihistamine; a container for containing the solution; and a transfer device, cooperative with the container, for transferring the solution to the lips of a person experiencing oral allergy symptoms.

In some embodiments, the transfer device is one of: a brush, a sponge, a roller ball, a hole sized to enhance capillary action, a nozzle.

Another general aspect of the invention is an applicator device for treatment of oral allergy symptoms of the lips. The applicator device includes: a lipstick incorporating at least one of: a mast cell stabilizer, a leukotriene inhibitor, and an antihistamine; and a lipstick dispenser for dispensing the lipstick.

### BRIEF DESCRIPTION OF THE DRAWINGS

The invention will be more fully understood from the following detailed description, in conjunction with the following figures, wherein:

FIG. 1 is a cross-sectional view of a roller lip applicator with a cap on, the applicator having an applicator ball;

FIG. 2 is a cross-sectional view of the lip applicator of FIG. 1, with the cap removed, the applicator being ready for use.

FIG. 3 is a cross-sectional view of a sponge lip applicator similar to the roller lip applicator shown in FIG. 1, having a sponge applicator instead of a applicator ball.

FIG. 4 is a cross-sectional view of the lip applicator of FIG. 3, with the cap removed, the applicator being ready for use.

FIG. 5 is a cross-sectional view of an aperture/plug lip applicator similar to the roller lip applicator shown in Fig. 1 and the sponge lip applicator shown in FIG. 3, the aperture lip applicator having a small aperture, and the cap having a protruding plug for sealing the aperture when the cap is on.

FIG. 6 is a cross-sectional view of the aperture/plug lip applicator of FIG. 5, with the cap removed, the aperture being open and ready for use. The aperture, when run across the lips, enables a small amount of the active ingredient to be applied.

FIG. 7 is a cross-sectional view of an aperture/O-ring lip applicator similar to aperture/plug lip applicator of FIG. 5, with an O-ring sealing the active liquid instead of the protruding plug.

FIG. 8 is a cross-sectional view of the aperture/O-ring lip applicator of FIG. 7, with the cap removed, the aperture being open and the applicator being ready for use.

FIG. 9 is a cross-sectional view of a cap-brush lip applicator having a brush on a long arm attached to a cap.

FIG. 10 is a cross-sectional view of the cap-brush lip applicator of FIG. 9, with the cap removed and ready for use.

FIG. 11 is a cross-sectional view of a cap-sponge lip applicator having a sponge swab on a long arm attached to a cap.

FIG. 12 is a cross-sectional view of the cap-sponge lip applicator of FIG. 11, with the cap removed and ready for use.

FIG. 13 is a cross-sectional view of a lipstick applicator with a cap.

FIG. 14 is a cross-sectional view of the lipstick applicator of FIG. 13, with the cap removed.

FIG. 15 is a cross-sectional view of the lipstick applicator of FIG. 13, with the cap removed and the applicator screw twisted so as to expose for use the active ingredient lipstick.

### DETAILED DESCRIPTION

Activation of mucosal mast cells in the oral cavity upon ingestion of raw fruits and/or raw vegetables may result in oral itchiness, that often precludes affected individuals from consuming raw fruits and vegetables. This may significantly adversely affect the allergic individual's quality of life, due to both personal and social implications, and it may compromise self-esteem especially in the pediatric age group, and may further raise nutritional concerns.

According to the invention, blocking the release of histamine and other mediators from the mast cells by mast cell stabilizers, such as Cromolyn Sodium, may enhance the ability of affected individuals to consume raw fruits and raw vegetables that otherwise would trigger an allergic reaction.

The proposed invention consists of introduction of a mast cell stabilizer, such as Cromolyn Sodium, prior to ingestion of fruits and vegetables, either through swallowing, e.g. as tablets, as capsules, or as a liquid or topical application, such as in the form of melting tabs, aerosol, oral wash, swabs, candy, or lollypop. Topical application of the mast cell stabilizer before consuming a food allergen, such as raw fruit or raw vegetable, will PREVENT or usefully minimize the uncomfortable oral allergic reaction, such as itching, swelling, etc.

Antihistamine (either first generation, or second generation) can be added to the topical formulation of the mast cell stabilizer, or it can replace it.

For example, to overcome its poor mucous-membrane absorption, Cromolyn Sodium may be mixed in a viscous solution as well as with mucoadhesive materials, such as cyclodextrins, chytosan , alginate, glycerine, propylene glycol so as to extend contact time between Cromolyn Sodium and the oral mucosa and increase the residence time of cromolyn on top of the mucosa by forming a coating over this membrane. Alternatively, or in combination with the foregoing, or Cromolyn Sodium may be mixed with drug absorption enhancers (e.g. EDTA, or Cyclodextrine or a surfactant—e.g polyoxyethelene 9-lauryl esther, Tween 60, poly acrylic acid, or bile acids—sodium cholate, sodium glycocholate, lauryl sulfate) or with carrier molecule which increase drug absorption, such as the carrier molecule, sodium N-(8 [2-hydroxybenzoyl]amino)caprylate, referred to as “SNAC”, to enhance its local and systemic absorption, and thereby maximize its effect.

Take the preparation of the invention five to fifteen minutes before eating a raw fruit that is expected to cause an oral allergy reaction. Take a test amount of the fruit to verify that no symptoms are experienced, i.e., that the oral allergic reaction has in fact been prevented.

An example of a safe and effective formulation is to combine 100 mg of Cromolyn Sodium, in 5 cc water (e.g. Gastrocrom™) with 100 mg of glycerin to create an oral solution. The glycerine helps the patient keep it in the mouth.

Alternatively, instead of glycerin alginate can be used as a 1% to 10% solution in water (e.g. 1 mg alginate/100 ml water)

Glycerol can also be used, usually in the 5-20% concentration range, and propylene glycol 1-10% can be used also. All are GRAS.

A flavoring and/or sweetener can be added to make the preparation more palatable.

Or, the mast cell stabilizer can be formulated as part of a mouth rinse, having generally the same surfactant properties as Anti-Plaque mouth rinse to help the preparation dwell longer in mouth.

Cromolyn Sodium must be taken into and topically applied to the oral mucosa so as to create a four-hour effectiveness window. Nevertheless, the preparation of the invention can be effective even when taken just a few minutes before eating fruit.

Cromolyn Sodium is poorly absorbed through mucosal barriers unless it is attached to a carrier molecule. Topical application of Cromolyn Sodium when attached to a carrier molecule is enhanced in effectiveness.

However, the polarity of the Cromolyn Sodium molecule may also interfere with its local and/or topical activity, and thus to maximize its effect, it may be embedded in a viscous solution, or it can be mixed with mucoadhesive.

Another benefit of the preparation of the invention is that swallowing this preparation is NOT needed, thereby reducing the amount of drug to which a patient would be exposed.

When using an anti-histamine either alone or in combination with a mast cell stabilizer, the anti-histamine should work fast, and be less sedating. The mast cell stabilizer selected should be the most effective mast cell inhibitor, i.e., a mast cell inhibitor of known efficacy in a variety of allergic conditions, with an excellent safety record, with minimal unpleasant taste. Also possibly combine with a taste masker, such as a flavor and/or a sweetener.

Suiteable Mast Cells Stabilizers Include:
Alamast™; Vistakon/Pemirolast potassium.
Alocril™; Allergan/Nedocromil sodium.
Alomide™; Alcon/Lodoxamide tromethamine.
Bepreve™; Ista/Bepotastine besilate.
Crolom™; Bausch & Lomb/Cromolyn sodium.
Elestat™; Inspire/Epinastein HCl.
Optivar™; Meda/Azelastine HCl.
Pataday™; Alcon/Olopatadine HCl.
Patanol™; Alcon/Olopatadine HCl.
Zaditor™; Novartis Ophthalmics OTC/Ketotifen (as fumarate).

The medications that are listed below are listed as Mast cell stabilizers for the eye.

Those that are also antihistamines are marked: “also an antihistamine.” These all can be used as mast cell stabilizers in preparations of the invention:
Zyrtec Itchy eye™; McNeil/ketotifen fumarate also an antihistamine.
Elestat™; Allergan/epinastine HCl also an antihistamine.
Claritin Eye™; Merck/Ketotifen also an antihistamine.
Alaway™; Bausch & Lomb/ketotifen fumarate also an antihistamine.
Alamast™; Vistakon/Pemirolast potassium.
Alocril™; Allergan/Nedocromil sodium.
Alomide™; Alcon/Lodoxamide tromethamine.
Bepreve™; Ista/Bepotastine besilate. also an antihistamine.
Crolom™; Bausch & Lomb/Cromolyn sodium.
Elestat™; Inspire/Epinastein HCl.
Optivar™; Meda/Azelastine HCl. also an antihistamine.
Pataday™; Alcon/Olopatadine HCl. also an antihistamine.
Patanol™; Alcon/Olopatadine HCl. also an antihistamine.
Zaditor™; Novartis Ophthalmics OTC/Ketotifen (as fumarate). also an antihistamine.

Below are medications that are listed only as antihistamines:
Emadine™; Alcon/Emedastine difumarate
Naphcon-A; Alcon/Naphazoline HCl.
Visin-A™; McNeil/Naphazoline HCl

The preparation can be applied topically as a mouthwash that includes glycerin.

It must stay in the mouth for a short while (15 to 90 seconds), long enough to be in contact with the oral mucosa and form a temporary coating on its surface. It will begin its effects within a short time after contact with the mucosa, about one to fifteen minutes.

The oral preparation can be used ad-hoc before ingestion of an allergenic food, or preventively, one to four times a day.

The preparation of the invention can also be applied topically as a mouth spray, or by a liposomal emulsion. A liposomal emulsion formulation easily enters the oral mucosa, thereby providing enhanced effectiveness. Much like human cells, liposomes encapsulate the active ingredients of the preparation within a lipid bilayer, thus enabling them to penetrate readily into the mucosa of the mouth.

Oral allergy can also occur upon consumption of raw nuts.

The pharynx and larynx can be involved with swelling and or itching.

The invention can also be used for prevention and/or treatment of minor local allergic reaction within the mouth to any food, such as cake that includes eggs and/or milk and/or soy and/or fish and/or shell-fish.

Oral allergy predominantly occurs when the fruits and vegetables are raw.

Topical antihistamines such as diphenhydramine (Benadryl) work well as an antihistamine incorporated into the preparation of the invention.

Local anesthetics for topical use such as benzocaine (currently used as a topical cream Lanacaine™ or Pramocaine™), can also advantageously be included in the preparation of the invention to further decrease discomfort due to oral allergy symptoms.

Counterirritants, such as mint oil, menthol, or camphor—can be advantageously included in preparations of the invention, alone, or in combination with other active ingredients therein, such as Cromolyn Sodium.

Flavoring compounds and/or taste-masking compounds can also be advantageously included in the preparation of the invention. Even a preparation of Cromolyn Sodium, water, and a flavoring compound (e.g., Strawberry, Cherry, Lemon, Mint) can be used as an effective mouth rinse or mouth wash for preventing and/or treating oral allergy symptoms. Also, a preparation of Cromolyn Sodium and a flavor masking compound to mask any unpleasant taste of the Cromolyn Sodium can be used as an effective mouth rinse or mouth wash for preventing and/or treating oral allergy symptoms.

Cromolyn Sodium: This drug has been approved by the U.S. Food & Drug Administration (FDA) for sale by prescription, and after decades of use, has an excellent safety record. Cromolyn Sodium in nasal form (NasalCrom™) is presently sold OTC in the USA. Cromolyn Sodium has been used for decades in treatment of multiple allergic conditions including asthma, but not yet for the prevention and/or treatment of oral allergy symptoms, including Oral Allergy Syndrome. Oral allergy symptoms include the oral itchiness associated with ingestion of raw fruits, such as raw apples, and raw vegetables, such as raw carrots. Side effects of Cromolyn Sodium are considered very uncommon and when swallowed, may include headache; GI upset; itchiness; irritability; rash; and muscle pain. Since the preparation of the invention provides oral exposure without swallowing the drug, side effects are expected to be even less common.

Cromolyn Sodium is a generic drug name for a mast cell stabilizer, and is sold under trade names that include: Gastrocrom™, Opticrom™, Crolom™, Nasalcrom™ and Intal™.

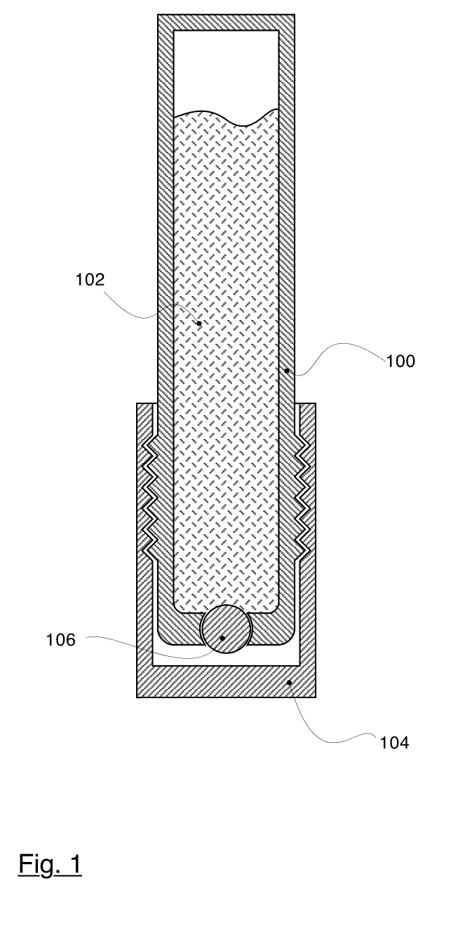

With reference to FIG. 1, a body 100 of a roller lip applicator contains an active ingredient 102. Active ingredients can include: a mast cell stabilizer and/or an antihistamine and/or a leukotriene inhibitor. For example, the mast cell stabilizer can be Cromolyn Sodium. The antihistamine can be levocetirizine (e.g. Xyzal™), or any other first, second, or third generation antihistamine. The leukotriene inhibitor can be montelukast (e.g., Singulair™). Cap 104 covers the applicator ball 106 when the lip roller applicator is not in use. When in use, the applicator ball 106 is able to roll in place when rolled in contact across the lips, without falling out of position. The applicator ball 106 has a surface that can transfer an effective amount of the active ingredient 102 to the lips. Effective amount depends on the active ingredient(s) used. For example, in the case of Cromolyn Sodium an effective amount is 0.1 ml of a solution of 100 mg of Cromolyn Sodium per 5 ml of water. In the case of antihistamines, e.g., levocitirizine, an effective amount is 0.1 ml of a solution of 0.5 mg levocitirizine per 1 ml water. In the case of leukotriene inhibitors, e.g., montelukast (e.g., Singulair™), an effective amount is 0.1 ml of a solution of 1 mg montelukast per 1 ml water.

A textured plastic ball can be used, such as a hollow polypropylene ball. The texture of the ball enhances the ability of the ball to roll over the surface of the lip and transfer the active ingredient 102 to the lips.

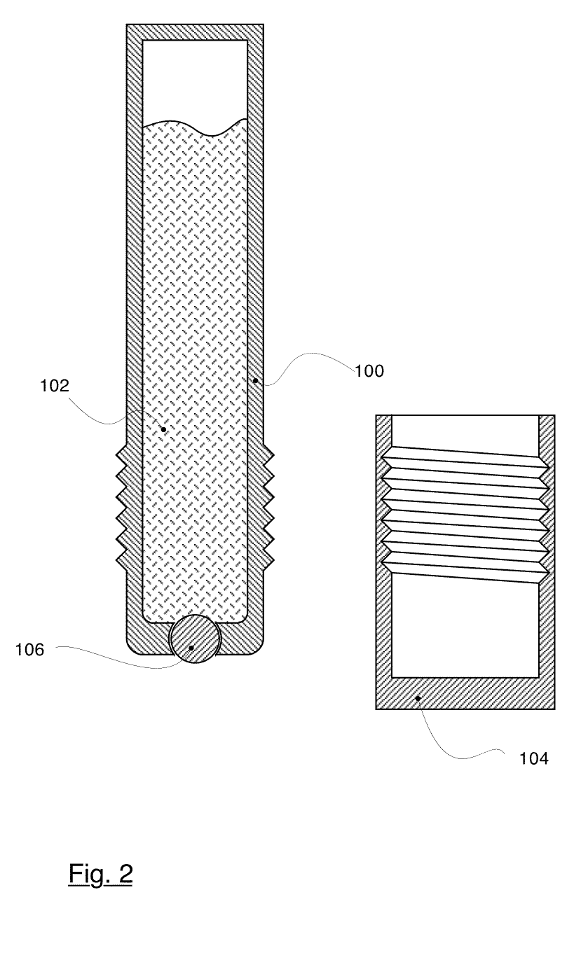

With reference to FIG. 2, the cap 104 has been unscrewed and removed from the body 100 to expose the applicator ball 106. The ball 106 transfers the active ingredient 102, which in this case contains a mast cell stabilizer such as Cromolyn Sodium, or an antihistamine such as levocetrizine, or a leukotriene inhibitor such as montelukast (e.g., Singulair™). The ball 106 is now ready to transfer the active ingredient 102 to the mucosal surface of the lip. The patient will hold the body 100 with his hand while the ball 106 at the tip is exposed and is rolled in a gentle smooth rolling movement across the mucosal surfaces of the upper lip and the lower lip. A patient diagnosed with oral allergies will be advised to use the device and apply the active ingredient 102 prior to ingesting the known allergen to which he or she is sensitive. Allergens can be fruits vegetables legumes and other food ingredients. The active ingredient 102, once in contact with superficial mast cells of the lips and basophils in the circulation, will now prevent the release of the powerful chemicals from granules inside these cells which may include histamine, leukotriens, and other allergy mediators which are the cause of the symptoms of oral allergies.

In case a patient exhibits symptoms of oral allergy after coming into contact or after ingesting a food allergen, it is not too late to apply the active ingredient 102. In fact, when the active ingredient is applied, symptoms are relieved promptly, i.e., within seconds. Thus, the active ingredient 102 can be use both as a preventive modality and as a treatment modality for oral allergies involving the oral mucosa and lips. After the application of the active ingredient 102 to the lips, the cap 104 is screwed back on to the body 100 so that the device remains clean and ready to be reused.

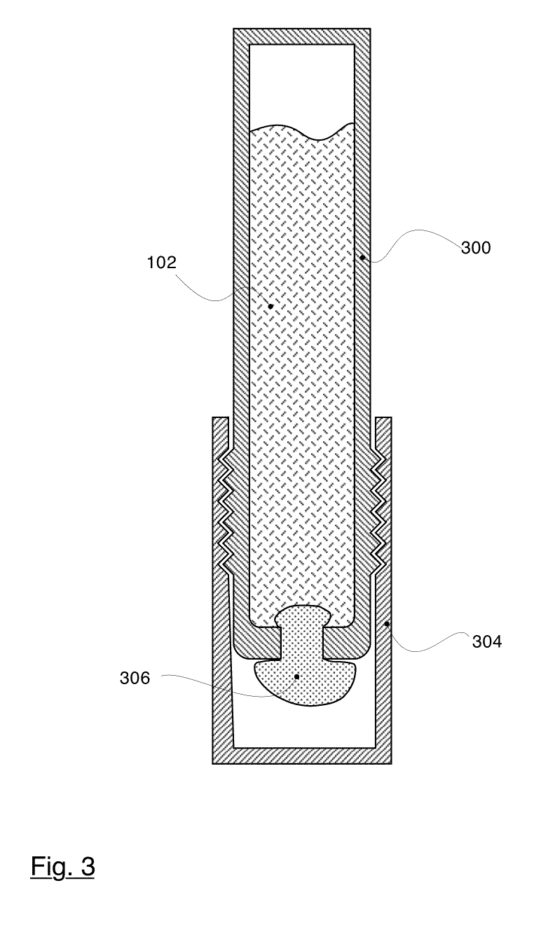

With reference to FIG. 3, a body 300 of a sponge lip applicator has a sponge 306 and contains an active ingredient 102. Cap 304 covers the sponge 306 when the lip applicator is not in use. When in use, the sponge 306 is able to transfer active ingredient 102 to the lips by gentle application across the lips, without falling out of position. A textured absorbent material, such as cotton, cloth, or a sponge can be used as the applicator 306. The material of the applicator 306 transfers active ingredient 102 to the surface of the lip.

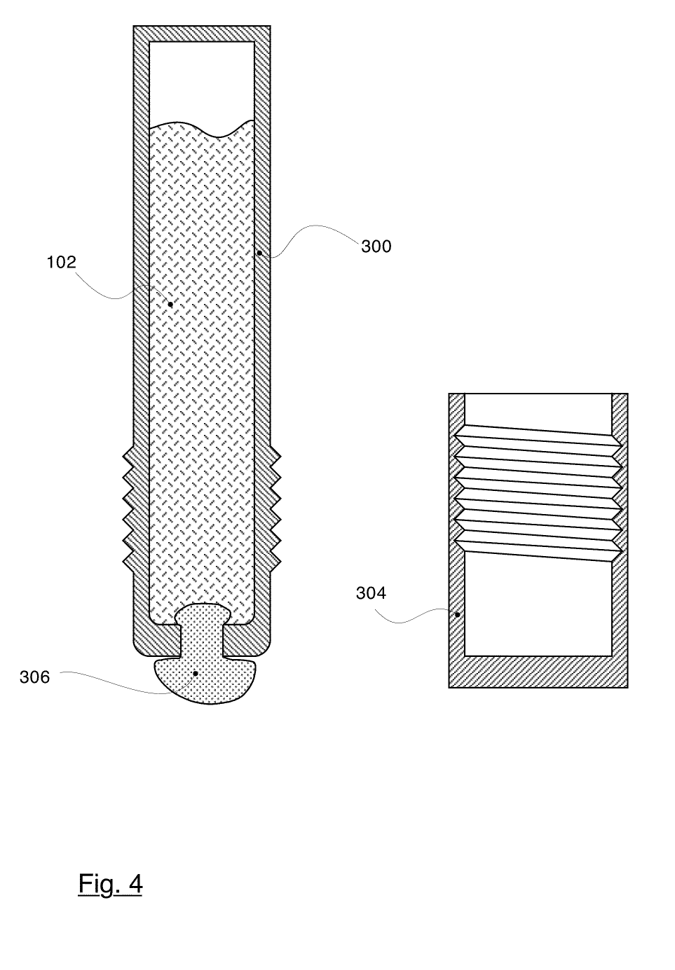

With reference to FIG. 4, the cap 304 has been unscrewed and removed from the body 300 to expose the sponge 306. Operation of the sponge lip applicator is similar to the operation of the lip roller applicator shown in FIG. 2. The sponge feels different from a roller when used to apply the active ingredient 102 to the lips, and it is a matter of personal preference as to which method, sponge or roller, is more comfortable.

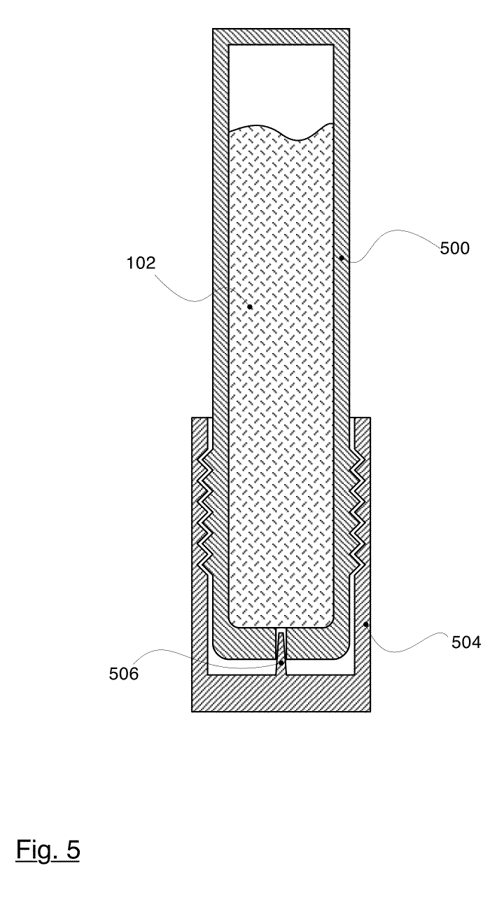

With reference to FIG. 5, an aperture/plug lip applicator contains active ingredient 102 inside body 500, and has a small aperture that can allow a slow flow of active ingredient 102 out of the body 500. As long as the cap 504 is screwed onto the body 500, the protruding plug 506, which is integral to the cover 504, seals the aperture and prevents leakage of active ingredient 102. To prevent leakage of active ingredient 102, it is important to properly seal the aperture by making sure that the cap 504 is screwed on all the way. Some very mild force might be required to ensure a proper seal.

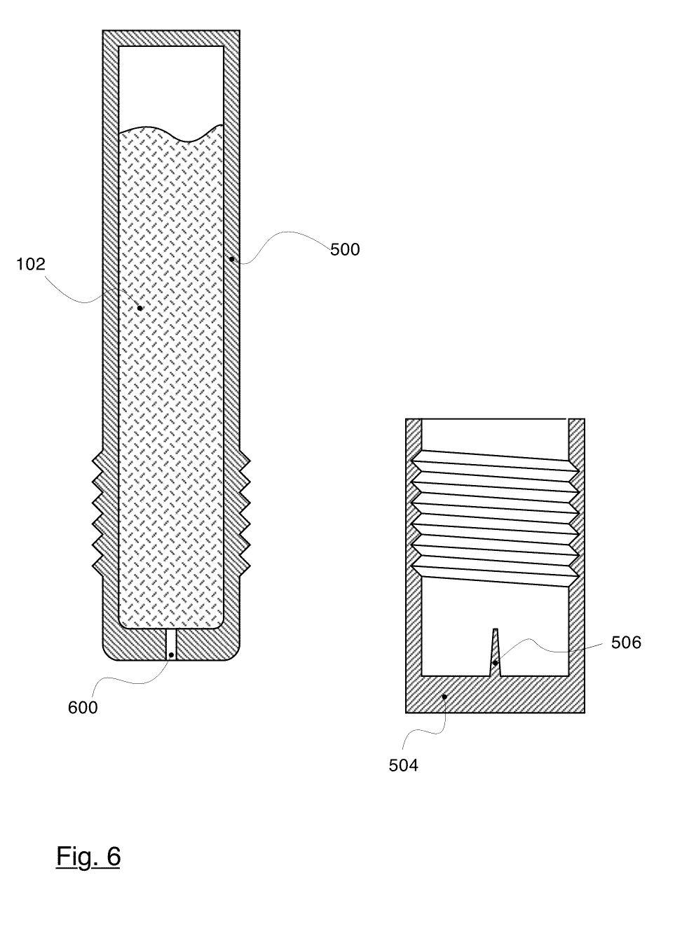

With reference to FIG. 6, the cap 504 has been unscrewed and removed from the body 500 to expose the aperture 600. The patient holds the body 500 with his/her hand while the aperture 600 at the tip is exposed and is glided in a gentle smooth motion across the mucosal surfaces of the upper lip and lower lip. The active ingredient 102 then flows through the aperture into a small area and will be spread around by the surface of the body 500 surrounding the aperture 600. The surface surrounding the aperture 600 will be wet, and therefore will be able to apply the active ingredient 102 to upper lip as well. When applying to the upper lip, depending on the desired amount of active ingredient, it may be necessary to repeatedly flip the aperture applicator to enable natural flow of active ingredient 102 onto the surface surrounding the aperture 600. After the application of the active ingredient 102, the cap 504 is screwed back onto the body 500, so that the device remains clean and ready to be reused. It is important to make sure that some mild force is used when screwing the cap 504 back onto the body 500, to ensure that the protruding plug 506 properly seals the aperture 600.

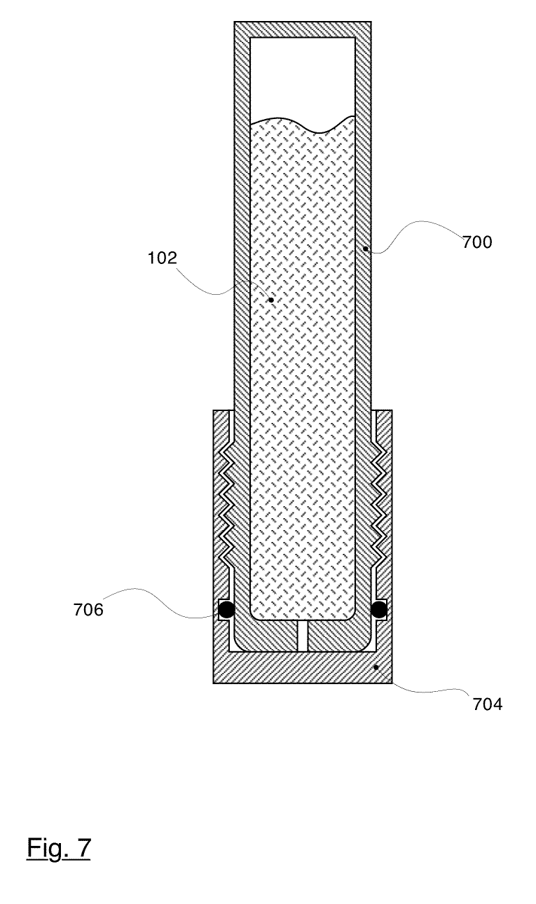

With reference to FIG. 7, the aperture/O-ring lip applicator is similar to the aperture/plug lip applicator shown in FIG. 5, but here the cap 704 has a groove and an O-ring 706 instead of a protruding plug. The O-ring provides a seal between the cap 704 and body 700, thereby preventing leakage of active ingredient 102 as long as the cap 704 is screwed on. Unlike the aperture/plug lip applicator, where the cap needs to be screwed on tightly, the quality of the seal provided by the O-ring does not depend on closing force. Therefore, there will be no need to ensure that the cap 704 is closed with any force beyond the minimal force that is required to screw on the cap 704. The advantage of an O-ring seal is that it requires very little force, and the seal is engaged as soon as the cap 704 is screwed even partially on. The disadvantage of the O-ring based seal is that it depends on the smoothness of the surface of the body 700 in the area that contacts the O-ring, and the smoothness of the outer wall of the groove in the cap 704 in which the O-ring is seated. There is no practical way to ensure a proper seal when the smoothness of the surfaces degrades enough to cause a leak. Closing the cap 704 tighter will not improve the quality of the seal, and a leaking aperture/O-ring lip applicator will need to be replaced.

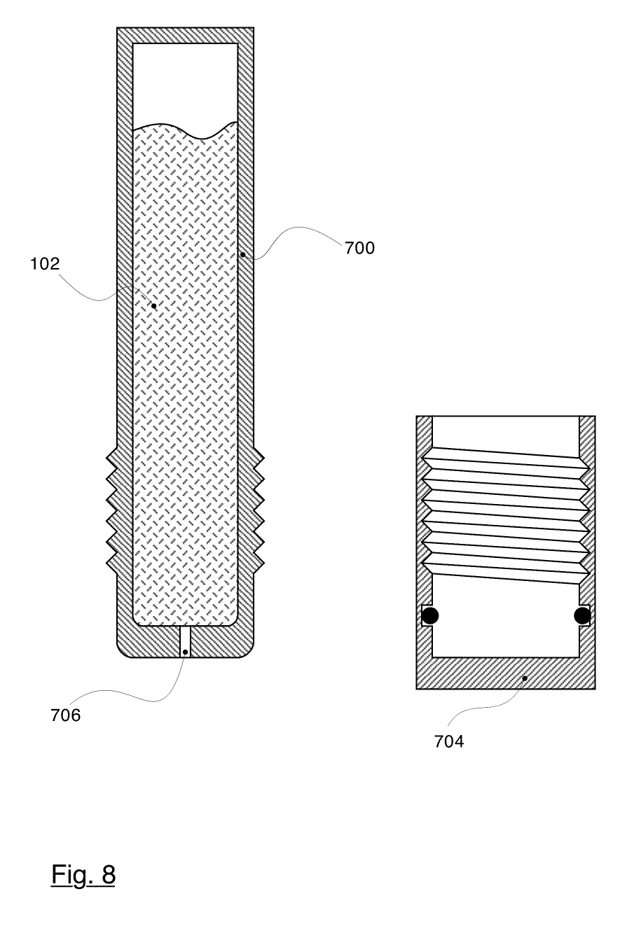

With reference to FIG. 8, the cap 704 has been unscrewed and removed from the body 700 to expose the aperture 706. The application of active ingredient 102 using aperture/O-ring lip applicator is similar to application of active ingredient using the aperture/plug lip applicator that is shown in FIG. 6. The difference is that in the case of the aperture/O-ring lip applicator, there is no need to use any significant force when screwing the cap 704 on after the application of the active ingredient 102 is completed. It is important to screw the cap 704 on to prevent leakage of the active ingredient 102 out of body 700, but there is no need to tighten beyond simply ensuring that the cap 704 is held in place, and will not fall off during regular handling such as carrying it in a pocket or storing it in a purse.

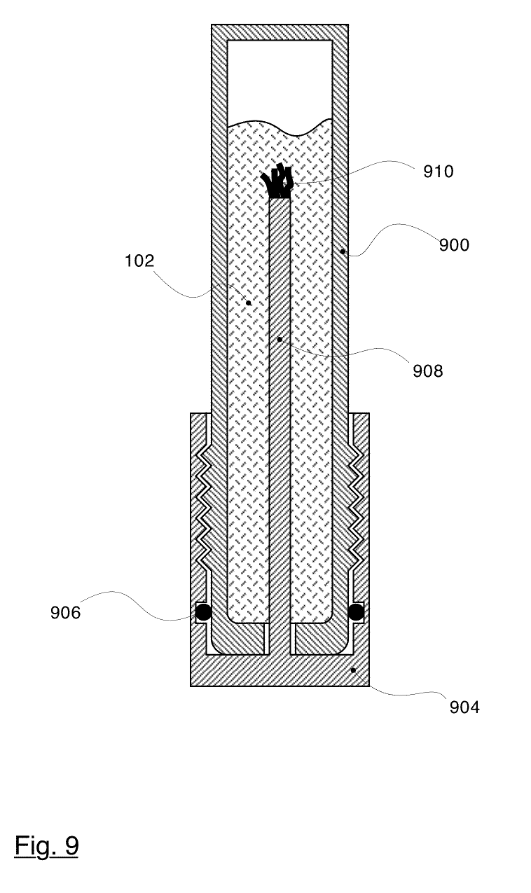

With reference to FIG. 9, a body 900 of a cap-brush lip applicator contains an active ingredient 102. Brush 904 is attached to a long arm 906 protruding from cap 908, while an 0-ring 910 seals the cap 908 to the body. Consequently the O-ring 910 prevents the active ingredient 102 from leaking out.

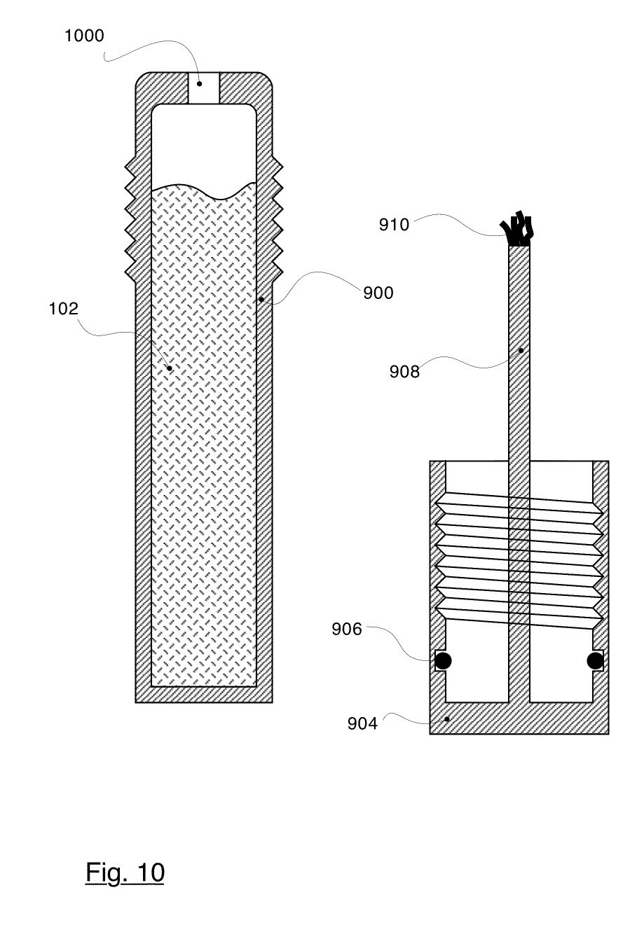

With reference to FIG. 10, to use the cap-brush lip applicator, the applicator must be held with the cap 908 facing up, in order to avoid spilling the active ingredient 102 while the cap 908 is being unscrewed. It is important to hold the body 900 with the opening 1000 facing up whenever the cap 908 is off. The cap 908 is separated from the body 900 to make it ready for use. The brush 904 is ready to be used to apply the active ingredient 102 to the lips. Applying an effective amount of active ingredient 102 is assured by, if necessary, repeatedly dipping the brush 904 and the arm 906 into body 900 through opening 1000. At the end of application, the brush 904 and the arm 906 are returned into body 900 through opening 1000, and the cap 908 is screwed back on. O-ring 910 provides a seal and prevents active ingredient 102 from leaking out, therefore the device no longer needs to be oriented with the opening 1000 facing up.

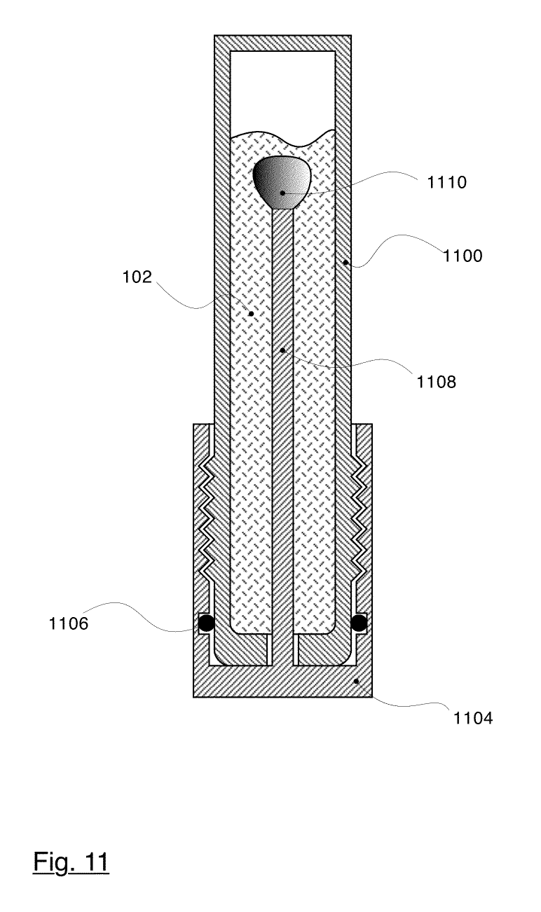

With reference to FIG. 11, a cap-sponge lip applicator is similar to the cap-brush lip applicator of FIG. 9. The only difference is that the cap-sponge lip applicator has a sponge 1100 instead of a brush attached to the end of the arm 1102.

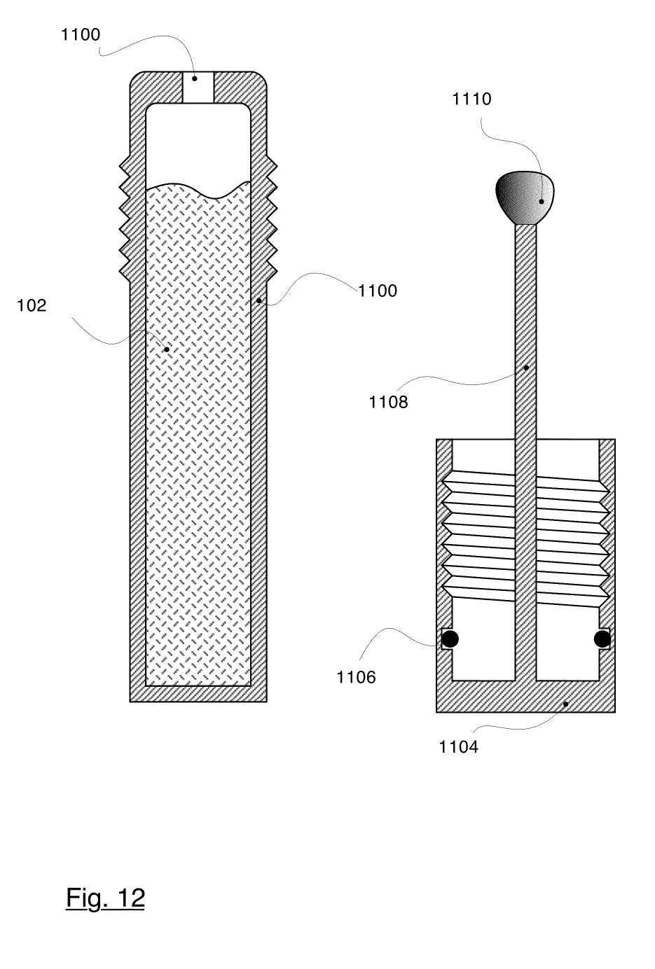

With reference to FIG. 12, this is the cap-sponge lip applicator of FIG. 11, with the cap 904 unscrewed, and the sponge 1100 ready to be used to apply the active ingredient 102 to the lips. The use of the cap-sponge lip applicator is similar to the use of the cap-brush lip applicator that is referenced in FIG. 10, the only difference being the sponge 1100 at the end of arm 1102 instead of a brush. The sponge 1100 can absorb more active ingredient than a brush, and therefore it may be used to apply more active ingredient 102 onto the lips between each dip. Also, the sponge feels different from a brush when used to apply the active ingredient 102 to the lips, and it is a matter of personal preference as to which method, sponge or brush, is more comfortable.

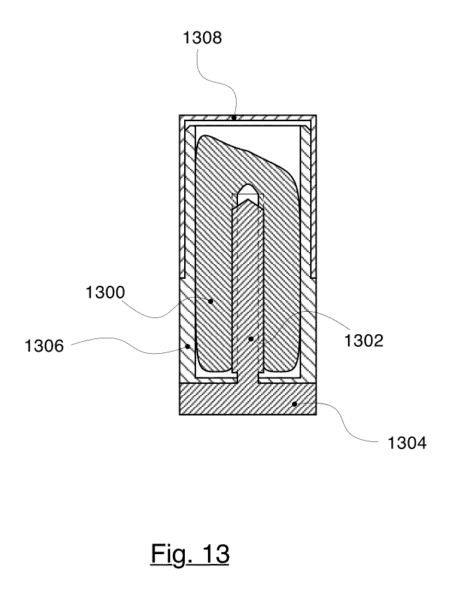

With reference to FIG. 13, a lipstick applicator is shown for use in a manner similar to known lipstick applicators, such as those used to apply cosmetics. Lipstick 1300, when applied to the lips, is capable of dispensing an effective amount of an active ingredient, such as the active ingredient 102. An example of an effective amount of an active ingredient, such as Cromolyn Sodium, is 2 mg of Cromolyn Sodium spread evenly over two lips. To make a lipstick 1300 capable of dispensing an effective amount per use of the lipstick 1300, standard known methods for making lipsticks are used, with the exception that a solution of active ingredient(s) is used instead of water alone. For example, in the case of Cromolyn Sodium, a solution of 100 mg of Cromolyn Sodium per 5 cc of water is used for the pre-emulsion instead of water alone.

Lipstick 1300 has an inner thread that is screwed onto applicator screw 1302 that is connected to handle 1304. The applicator screw 1302 together with the handle 1304 are free to rotate about their natural axis relative to body 1306 without disengaging from body 1306. The lipstick 1302 can move up and down within the body 1300, when moved by the applicator screw 1302, but is mechanically constrained so as to not rotate along its axis. Cap 1308 has a snug fit to body 1306 so that a reasonably mild force is required to slide the cap 1308 off body 1306. The required force should be low enough to enable easy removal, yet sufficient to hold the cap 1308 in place and prevent unintentional removal during casual handling, such as placing the lipstick in or removing the lipstick from a pocket, or storing the lipstick in a purse. It is also possible to introduce a slight thread to the outer surface of the body 1306 and a matching thread to the corresponding inner surface of the cap 1308 so that removal of the cap 1308 would require a slight twist.

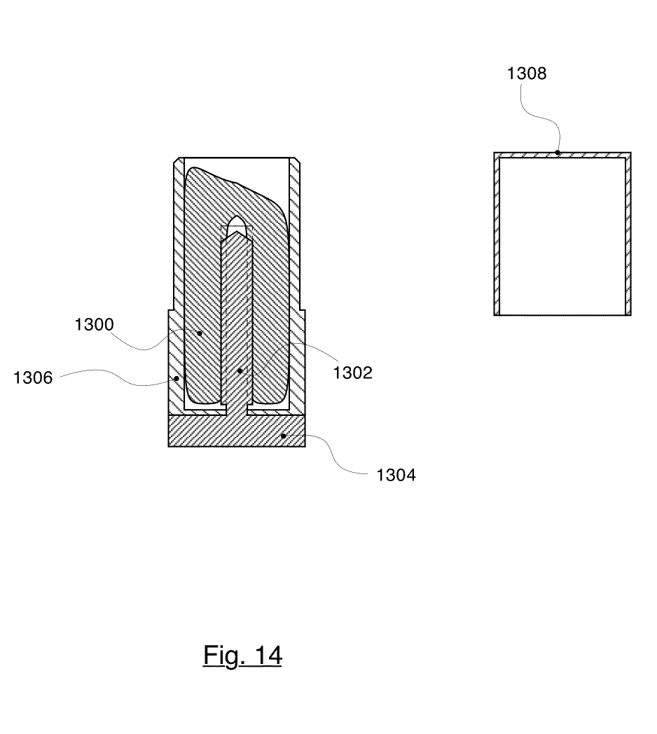

With reference to FIG. 14, this is the lipstick applicator with cap 1308 removed. To remove the cap 1308, a patient holds the lipstick applicator in one hand and removes the cap 1308 with the other hand. Removing the cap 1308 enables operation of the handle 1304, but does not yet expose lipstick 1300, as it is contained completely within the body 1306.

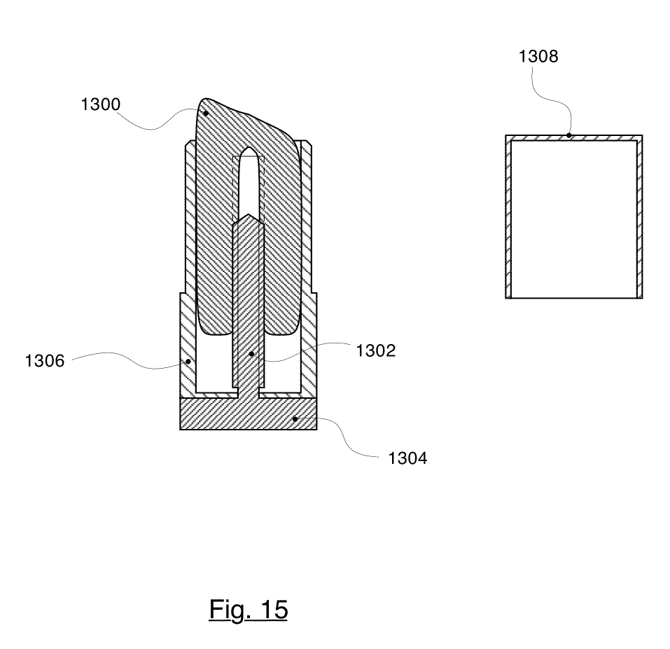

With reference to FIG. 15, the patient holds the body 1306 with one hand and twists handle 1304 with his/her other hand. Twisting the handle 1304 also twists the applicator screw 1302. The lipstick 1300 cannot rotate and the applicator screw cannot disengage from the body 1306, therefore the thread on the applicator screw 1302 pushes the lipstick 1300 out of the body 1306. Handle 1304 is twisted until enough lipstick is exposed to conveniently apply it along the exterior and, if necessary, interior of the lips in order to transfer an effective amount of active ingredient. When application of the active ingredient in the lipstick 1300 is completed, the patient holds the body 1306 in one hand and twists the handle 1304, with the other hand, in the opposite direction until the lipstick 1300 is completely retracted into the body 1306. The patient then continues holding the body 1306 with one hand and replaces the cap 1308 with the other hand, applying slight pressure to ensure a snug fit. If the cap 1308 has a slight thread on it, then fitting the cap 1308 back onto the body 1306 will require a slight twist in the opposite direction from what was used to remove the cap 1308 from the body 1306.

Active ingredient(s) can also be applied to the lips as a lotion, a cream, an ointment, or a gel. In each case, one of average skill in the art would know how to formulate each so as to deliver an effective amount per dose. Effective amount depends on the active ingredient(s) used. For example, in the case of Cromolyn Sodium an effective amount is 0.1 ml of a solution of 100 mg of Cromolyn Sodium per 5 ml of water. In the case of antihistamines, e.g., levocitirizine, an effective amount is 0.1 ml of a solution of 0.5 mg levocitirizine per 1 ml water. In the case of leukotriene inhibitors, e.g., montelukast (e.g., Singulair™), an effective amount is 0.1 ml of a solution of 1 mg montelukast per 1 ml water.

Active ingredients can also be applied to the lips in the form of a lip gloss that imparts gloss and/or color to the lips.

Some formulations of the active ingredients can have a taste that is objectionable or unpleasant to some people. For example, some people experience a slightly bitter taste after administration of Cromolyn Sodium. Unpleasant tastes of medications can be masked or blocked. Thus, it may be advantageous to add to some active ingredients and/or a delivery medium a taste-masking or taste-blocking compound.

Some formulations of the active ingredients benefit from the addition of coloring agents that enhance the subjective and/or aesthetic experience of the patient.

Other modifications and implementations will occur to those skilled in the art without departing from the spirit and the scope of the invention as claimed. Accordingly, the above description is not intended to limit the invention, except as indicated in the following claims.

What is claimed is:

1. A method for the treatment and/or prevention of oral allergic symptoms of the lips due to oral contact with a food allergen, the method comprising:
topically administering to the lips an effective amount of at least one of:
a mast cell stabilizer;
an antihistamine; and
a leukotriene inhibitor.

2. The method of claim 1, wherein the mast cell stabilizer also has anti-histamine properties.

3. The method of claim 1, wherein, to extend contact time of the at least one of the mast cell stabilizer and the antihistamine with the lip, at least one of the mast cell stabilizer and the antihistamine is mixed with at least one of:
an oily carrier; and
mucoadhesive material.

4. The method of claim 1, wherein the mast antihistamine is levocetirizine.

5. The method of claim 1, wherein the leukotriene inhibitor is montelukast.

6. The method of claim 1, wherein the mast cell stabilizer is mixed with a carrier molecule so as to enhance local absorption.

7. The method of claim 1, wherein an effective amount of at least one of a mast cell stabilizer and an antihistamine is applied by at least one of:
a lip swab, a lipstick, a lip roller, a lip brush, a sponge, a tube, a jar.

8. The method of claim 1 wherein the effective amount of at least one of: a mast cell stabilizer, an antihistamine, and a leukotriene inhibitor, is contained within a lipstick and is topically administered to the lips of a person experiencing oral allergy symptoms by application of the lipstick thereto, the lipstick being contained within and dispensed from a lipstick dispenser.

9. A method for the treatment and/or prevention of oral allergic symptoms of the lips due to oral contact with a food allergen, the method comprising:
topically administering to the lips an effective amount of Cromolyn Sodium.

10. The method of claim 9 wherein the effective amount of Cromolyn Sodium is held in a container and is topically administered to the lips of a person experiencing oral allergy symptoms by a transfer device, cooperative with the container.

11. The method of claim 10, wherein the transfer device is one of:
a brush, a sponge, a roller ball, a hole sized to enhance capillary action, a nozzle.

12. The method of claim 9 wherein the effective amount of Cromolyn Sodium is contained within a lipstick and is topically administered to the lips of a person experiencing oral allergy symptoms by application of the lipstick thereto, the lipstick being contained within and dispensed from a lipstick dispenser.

13. The method of claim 9, wherein, to extend contact time of the Cromolyn Sodium with the lip, the Cromolyn Sodium is mixed with a mucoadhesive material.

14. The method of claim 9, wherein, to extend contact time of the Cromolyn Sodium with the lip, the Cromolyn Sodium is mixed with an oily carrier.

15. The method of claim 1, wherein the Cromolyn Sodium is mixed with a carrier molecule so as to enhance local absorption.

16. The use of Cromolyn Sodium in the manufacture of a medicament for the topical treatment and/or topical prevention of oral allergic symptoms of the lips due to oral contact with a food allergen.
